(19) United States
(12) Patent Application Publication
(10) Pub. No.: US 2023/0340099 A1
(43) Pub. Date: Oct. 26, 2023

(54) COMPOSITION COMPRISING CHEMOKINE INHIBITOR, COLONY STIMULATING FACTOR INHIBITOR, AND CANCER IMMUNOTHERAPY AGENT FOR PREVENTION OR TREATMENT OF CANCER AND COMBINATION THERAPY

(71) Applicant: AJOU UNIVERSITY INDUSTRY-ACADEMIC COOPERATION FOUNDATION, Suwon-si, Gyeonggi-do (KR)

(72) Inventors: Tae-Jun PARK, Seongnam-si (KR); Jang-Hee KIM, Suwon-si (KR); Yong-Won CHOI, Yongin-si (KR)

(73) Assignee: AJOU UNIVERSITY INDUSTRY-ACADEMIC COOPERATION FOUNDATION, Suwon-si, Gyeonggi-do (KR)

(21) Appl. No.: 17/797,916

(22) PCT Filed: Feb. 8, 2021
(86) PCT No.: PCT/KR2021/001645

§ 371 Date: Aug. 5, 2022

(30) Foreign Application Priority Data
Feb. 6, 2020 (KR) 10-2020-0014454

(51) Int. Cl. C07K 16/24; A61P 35/00; C07K 16/28; G01N 33/574

(52) U.S. Cl. CPC C07K 16/24; A61P 35/00; C07K 16/2818; C07K 16/243; G01N 33/57419; A61K 2039/507

(57) ABSTRACT

A composition containing a chemokine inhibitor, a colony stimulating factor inhibitor, and an cancer immunotherapy agent, and a method for treatment of cancer, and a combination therapy are disclosed. The composition containing a CXCL12 inhibitor, a colony stimulating factor 1 (CSF1) inhibitor, and a cancer immunotherapy agent, for prevention and/or treatment of cancer, increases the infiltration and activity of CD8 cytotoxic T cells in cancers resistant to cancer immunotherapy agents, especially PD-1/PD-L1 immune checkpoint inhibitors, to effectively reduce tumor sizes and thus is useful for prevention and treatment of cancers resistant to immunotherapy agents, especially cancers overexpressing p16^{INK4A}.

### TECHNICAL FIELD

The present invention relates to a composition for the treatment of cancer including a chemokine inhibitor, a colony-stimulating factor inhibitor, and a cancer immunotherapy agent, and combination therapy, and more particularly to a pharmaceutical composition for the prevention and/or treatment of cancer including a CXCL12 inhibitor, a CSF1 (colony-stimulating factor 1) inhibitor, and a cancer immunotherapy agent.

### BACKGROUND ART

Existing anticancer drugs include chemotherapy agents (cytotoxic anticancer agents) called first-generation anticancer agents that attack cancer cells by toxifying them with drugs, or targeted therapy agents called second-generation anticancer agents that selectively attack only specific targets of cancer cells. These existing anticancer drugs have side effects such as attacking normal cells as well as cancer cells or deteriorating efficacy thereof due to resistance.

Recently, with the goal of solving these problems and effectively treating cancer, research is ongoing to overcome the limitations of cancer treatment by regulating the immune system, such as inducing immune cells of a patient to attack cancer cells, rather than directly attacking cancer cells with chemical drugs. Cancer immunotherapy agents developed based on these studies are therapeutic agents having a mechanism of restoring or strengthening the tumor recognition or destruction ability of the immune system in order to overcome the acquired immunosuppression or immune evasion mechanism in cancer cells. These cancer immunotherapy agents include immune checkpoint inhibitors, immune cell therapy agents, immune virus therapy agents, and the like.

Immune checkpoint inhibitors are cancer immunotherapy agents capable of replacing existing anticancer drugs, and maximally activate the immune function of the body's immune cells for a certain period of time, such as activating or deactivating the immune function of the body's immune cells, etc., thereby maximizing the body's defense function. For example, PD-1 is an immune checkpoint protein of CD8 T cells, and modulates the immune activity of T cells. PD-L1, which is a ligand of PD-1, is overexpressed in many types of epithelial cancer, and when PD-L1 binds to the receptor PD-1 of CD8 T cells, the ability of CD8 T cells to attack cancer cells is deteriorated. Here, when an immune checkpoint inhibitor is administered, CD8 T cells are reactivated, thereby inducing the death of cancer cells.

However, in the case of immune checkpoint inhibitor monotherapy, many patients do not respond to treatment due to intrinsic resistance or congenital resistance, or a problem in that acquired resistance is strengthened after exhibiting an effect at the onset of treatment occurs. In some cases of colorectal cancer, resistance to treatment with such an immune checkpoint inhibitor appears, which causes problems such as decreased CD8 T-cell infiltration in tumors, and the development of various methods to overcome resistance is required.

In recent years, many attempts have been made to overcome resistance to cancer immunotherapy agent, including in particular immune checkpoint inhibitor monotherapy. For example, Korean Patent Application No. 10-2019-7008792 discloses combination therapy using an anti-PD-1 antibody, a STING agonist, an IL-15 super agonist, and an anti-CD40 antibody, and WO2016-201425 discloses combination therapy using an immune checkpoint inhibitor and a CXCR4 antibody. However, there are still very few alternative methods or combination therapies for overcoming resistance to immune checkpoint inhibitor monotherapy.

Meanwhile, in the case of treatment using cancer immunotherapy, besides the development of various therapeutic regimens, the use of therapy optimized for the type of cancer that the patient is actually suffering from is regarded as important. Cancer occurs due to various causes, and even cancer occurring in the same bodily region shows different characteristics such as metastasis, resistance, etc., depending on the location of the mutation, the tumor microenvironment, and the like. In particular, in order to overcome monotherapy resistance in cancer immunotherapy, various combination therapies are being researched and developed. However, in order to obtain a sufficiently powerful effect, it is very important to identify the molecular biological basis for the combination therapy, and also to discriminate various markers and characteristics of cancer and to develop and apply an optimized therapy.

As described above, the cancer immunotherapy agent shows an ‘all-or-nothing’ type of treatment response in that, when effective, a sufficiently strong effect is exhibited, thereby realizing a high treatment rate, but in other cases there is absolutely no effect. Almost no methods for predicting treatment response or treatment resistance have been reported, and in particular, no relationship between expression of p16^{INK4A} in cancer cells and immunotherapy resistance has been reported.

Korean Patent Application No. 10-2019-0002873 authored by the present inventors (unpublished) discloses a biomarker for predicting resistance to treatment using a cancer immunotherapy agent by confirming overexpression of CXCL12 and CSF1 genes in p16^{INK4A}-overexpressing colorectal cancer and revealing the mechanism of resistance to a cancer immunotherapy agent due to overexpression of the corresponding genes.

Against this background, the present inventors have made great efforts to effectively treat cancer that is resistant to cancer immunotherapy agents, and ascertained that CXCL12 and CSF1 genes are overexpressed in p16^{INK4A}-overexpressing colorectal cancer and overexpression of the corresponding genes inactivates T cells through reduction of T-cell tumor infiltration and activity, whereby cancer becomes resistant to cancer immunotherapy agents, and also that, based on the mechanism of acquired treatment resistance, combination therapy using a CXCL12 inhibitor, a CSF1 inhibitor, and a cancer immunotherapy agent exhibits a remarkable effect in the treatment of cancer, particularly cancer that is resistant to cancer immunotherapy agents, through a synergistic effect, thus culminating in the present invention.

### DISCLOSURE

It is an object of the present invention to provide a pharmaceutical composition for the prevention and/or treatment of cancer, including a CXCL12 (C-X-C motif chemokine 12) inhibitor, a CSF1 (colony-stimulating factor 1) inhibitor, and a cancer immunotherapy agent.

It is another object of the present invention to provide combination therapy of a CXCL12 (C-X-C motif chemokine 12) inhibitor and a CSF1 (colony-stimulating factor 1) inhibitor with a cancer immunotherapy agent or immunotherapy.

It is still another object of the present invention to provide the use of a CXCL12 inhibitor and a CSF1 inhibitor for co-administration with a cancer immunotherapy agent.

It is yet another object of the present invention to provide a method of preventing and/or treating cancer including administering a CXCL12 inhibitor, a CSF1 inhibitor, and a cancer immunotherapy agent to a subject.

It is still yet another object of the present invention to provide a method of preventing and/or treating cancer, including:
measuring the expression or activity level of at least one gene selected from the group consisting of p16^{INK4A}, CXCL12, and CSF1 or a protein encoded thereby in a sample isolated from a subject;
predicting cancer immunotherapy agent resistance of the subject by comparing the expression or activity level of the gene or the protein encoded thereby with the expression or activity level of a corresponding gene or a protein encoded thereby in a normal control sample; and
co-administering a CXCL12 inhibitor, a CSF1 inhibitor, and a cancer immunotherapy agent when the expression or activity level of the gene or the protein encoded thereby is increased.

In order to accomplish the above objects, the present invention provides a pharmaceutical composition for the prevention and/or treatment of cancer including a CXCL12 (C-X-C motif chemokine 12) inhibitor, a CSF1 (colony-stimulating factor 1) inhibitor, and a cancer immunotherapy agent.

In addition, the present invention provides combination therapy of a CXCL12 (C-X-C motif chemokine 12) inhibitor and a CSF1 (colony-stimulating factor 1) inhibitor with a cancer immunotherapy agent or immunotherapy.

In addition, the present invention provides the use of a CXCL12 inhibitor and a CSF1 inhibitor for co-administration with a cancer immunotherapy agent for the prevention and/or treatment of cancer.

In addition, the present invention provides the use of a CXCL12 inhibitor and a CSF1 inhibitor for the prevention and/or treatment of cancer through co-administration with a cancer immunotherapy agent.

In addition, the present invention provides the use of a CXCL12 inhibitor, a CSF1 inhibitor, and a cancer immunotherapy agent for the manufacture of a pharmaceutical composition for co-administration for the prevention and/or treatment of cancer.

In addition, the present invention provides a method of preventing and/or treating cancer including co-administering a CXCL12 inhibitor, a CSF1 inhibitor, and a cancer immunotherapy agent to a subject.

In addition, the present invention provides a method of preventing and/or treating cancer, including:
measuring the expression or activity level of at least one gene selected from the group consisting of p16^{INK4A}, CXCL12, and CSF1 or a protein encoded thereby in a sample isolated from a subject;
predicting cancer immunotherapy agent resistance of the subject by comparing the expression or activity level of the gene or the protein encoded thereby with the expression or activity level of a corresponding gene or a protein encoded thereby in a normal control sample; and
co-administering a CXCL12 inhibitor, a CSF1 inhibitor, and a cancer immunotherapy agent when the expression or activity level of the gene or the protein encoded thereby is increased.

### DESCRIPTION OF DRAWINGS

FIG. 1 shows results of immunohistochemistry staining of p16^{INK4A} in colorectal cancer samples according to Example 1,

FIG. 1A showing representative immunohistochemistry staining images of tissues classified depending on the proportion of stained cells (0: less than 1%; 1+: 1-20%; 2+: 20-40%; and 3+: greater than 40%),

FIG. 1B showing results of classification of 120 samples;

FIG. 2 shows results confirming the expression of CXCL12 and CSF1 in p16^{INK4A}-overexpressing colorectal cancer through immunochemistry staining,

FIG. 2A showing staining images of p16^{INK4A} and CXCL12 confirmed through immunochemistry staining,

FIG. 2B showing staining images of p16^{INK4A} and CSF1 confirmed through immunochemistry staining,

FIG. 2C being a graph showing changes in expression of CSF1 depending on the expression level of p16^{INK4A};

FIG. 3A shows photographs taken by subcutaneously injecting control tumor cells (MC38) and CXCL12-overexpressing tumor cells (MC38-mCXCL12) into mice and isolating the tumor cells after 3 weeks;

FIG. 3B is graphs showing the tumor sizes of control tumor cells (MC38) and CXCL12-overexpressing tumor cells (MC38-mCXCL12) in mice (N=10) (upper) and a normalized graph (lower), each value in the lower graph being mean±SD;

FIG. 3C shows results confirming the infiltration of CD8 T cells in control tumor cells (MC38) and CXCL12-overexpressing tumor cells (MC38-mCXCL12) in the upper images, black arrows representing CD8 T cells,
the lower graph of FIG. 3C showing the rates of growth of two cell groups when control tumor cells (MC38) and CXCL12-overexpressing tumor cells (MC38-mCXCL12) were cultured in vitro in cell culture dishes;

FIG. 3D shows the counted number of CD8 T cells infiltrated into control tumor cells (MC38) and CXCL12-overexpressing tumor cells (MC38-mCXCL12), each black bar representing the average value;

FIG. 4A shows images confirming the intratumoral infiltration of CD8 T cells in CXCL12-overexpressing tumor cells (MC38-mCXCL12) when a CSF1 antibody, a PD-1 antibody, and a CSF1 antibody were administered alone or in combination;

FIG. 4B shows the number of CD8 T cells infiltrated into CXCL12-overexpressing tumor cells (MC38-mCXCL12) when a CSF1 antibody, a PD-1 antibody, and a CSF1 antibody were administered alone or in combination;

FIG. 5 shows the results of ELISA, immunostaining, and FACS analysis confirming the induction of monocyte differentiation of CSF1;

FIG. 6A is photographs and graphs showing the tumor cell sizes observed after subcutaneous injection of control tumor cells (MC38), CSF1-overexpressing tumor cells (MC38-CSF1), and CSF1-inhibiting tumor cells (MC38-CSF1) into mice;

FIG. 6B shows the results of analysis of the differentiation of M2-type macrophages (CD206+) in the tissues of control tumor cells (MC38), CSF1-overexpressing tumor cells (MC38-CSF1), and CSF1-inhibiting tumor cells (MC38-CSF1) and the number of granzyme B (GZMB) positive T cells through immunochemistry staining and ELISA;

FIG. 7A schematically shows an animal experimental method for confirming tumor size reduction by single administration or co-administration of the PD-1 antibody, the CXCL12 antibody, and the CSF1 antibody;

FIG. 7B is photographs showing the tumor cell sizes when the PD-1 antibody, the CXCL12 antibody, and the CSF1 antibody were administered alone or in combination to mice injected with CXCL12-overexpressing tumor cells (MC38-mCXCL12) (n=10);

FIG. 7C is graphs showing the tumor cell sizes when the PD-1 antibody, the CXCL12 antibody, and the CSF1 antibody were administered alone or in combination to mice injected with CXCL12-overexpressing tumor cells (MC38-mCXCL12) (n=10);

FIG. 7D is a graph showing the average tumor cell sizes when the PD-1 antibody, the CXCL12 antibody, and the CSF1 antibody were administered alone or in combination to mice injected with CXCL12-overexpressing tumor cells (MC38-mCXCL12) (n=10), each value being mean±SD; and

FIG. 8 is a graph showing the results of calculation of the tumor cell size reduction (%) when the PD-1 antibody, the CXCL12 antibody, and the CSF1 antibody were administered alone or in combination to mice injected with CXCL12-overexpressing tumor cells (MC38-mCXCL12) (n=10) compared to a control (IgG), each value being mean±SD.

### MODE FOR INVENTION

Unless otherwise defined, all technical and scientific terms used herein have the same meanings as those typically understood by those skilled in the art to which the present invention belongs. In general, the nomenclature used herein is well known in the art and is typical.

Cancer cells have a mechanism enabling them to evade detection by the body's immune system. Initially, the immune system may recognize and attack cancer cells, but at a certain point in time, the cancer cells grow and spread to other parts of the body by avoiding being attacked by the immune system through the immune surveillance evasion mechanism. A cancer immunotherapy agent inhibits the evasion of cancer cells from the body's immune system or strengthens the action of immune cells so that immune cells are capable of attacking cancer cells more effectively. Cancer immunotherapy agents are called third-generation anticancer agents, following first-generation (chemotherapy) anticancer agents and second-generation (targeted therapy) anticancer agents, and are advantageous in that there are few side effects of anticancer agents and in that the survival period is long due to the use of the patient's immune system.

However, when such a cancer immunotherapy agent is used alone, many patients do not respond to treatment due to intrinsic resistance or congenital resistance, or there is a problem in that acquired resistance is strengthened after an effect is observed at the onset of treatment. It is necessary to develop alternative therapy or combination therapy in order to overcome the above problems.

In an embodiment of the present invention, the present inventors confirmed that the expression of the CXCL12 gene and the CSF1 gene was greatly increased at the same time in colorectal cancer in which p16^{INK4A} was overexpressed. In another embodiment, overexpression of the CXCL12 gene and the CSF1 gene and the mechanism of acquired resistance to a cancer immunotherapy agent were revealed. Correspondingly, among currently useful immune checkpoint inhibitors, such as CTLA-4 inhibitors, PD-1 inhibitors, and PD-L1 inhibitors, there is no immune checkpoint inhibitor for colorectal cancer as the main indication.

In a further embodiment of the present invention, based on the mechanism of acquired resistance to a cancer immunotherapy agent due to the increased expression of CXCL12 and CSF1 in cancer cells, the expression inhibitor of each gene and the immune checkpoint inhibitor were administered in combination, confirming the therapeutic effect on the corresponding cancer cells. When the CXCL12 antibody, the CSF1 antibody, and the immune checkpoint inhibitor (PD-1 antibody) were administered in combination, the tumor cell size reduction effect was improved by 330% or more compared to when the immune checkpoint inhibitor was administered alone, and by 150% or more compared to when the immune checkpoint inhibitor and CXCL12 were administered in combination, confirming that the tumor size was reduced by 70%-99% and also that co-administration of a CXCL12 inhibitor, a CSF1 inhibitor, and a cancer immunotherapy agent showed a dramatic improvement in the therapeutic effect on cancer resistant to cancer immunotherapy agents through a synergistic effect.

Accordingly, an aspect of the present invention pertains to a pharmaceutical composition for preventing and/or treating cancer, including a CXCL12 (C-X-C motif chemokine 12) inhibitor, a CSF1 (colony-stimulating factor 1) inhibitor, and a cancer immunotherapy agent.

Another aspect of the present invention pertains to the use of a CXCL12 inhibitor, a CSF1 inhibitor, and a cancer immunotherapy agent for the manufacture of a pharmaceutical composition for co-administration for the prevention and/or treatment of cancer.

Still another aspect of the present invention pertains to combination therapy of a CXCL12 (C-X-C motif chemokine 12) inhibitor and a CSF1 (colony-stimulating factor 1) inhibitor with a cancer immunotherapy agent or immunotherapy.

Yet another aspect of the present invention pertains to the use of a CXCL12 inhibitor and a CSF1 inhibitor for co-administration with a cancer immunotherapy agent.

Still yet another aspect of the present invention pertains to the use of a CXCL12 inhibitor and a CSF1 inhibitor for the prevention and/or treatment of cancer through co-administration with a cancer immunotherapy agent.

A further aspect of the present invention pertains to a method of preventing and/or treating cancer including administering a CXCL12 inhibitor, a CSF1 inhibitor, and a cancer immunotherapy agent to a subject.

Still a further aspect of the present invention pertains to a method of preventing and/or treating cancer, including:
(a) measuring the expression or activity level of at least one gene selected from the group consisting of p16^{INK4A}, CXCL12, and CSF1 or a protein encoded thereby in a biological sample isolated from a subject;
(b) predicting cancer immunotherapy agent resistance of the subject by comparing the expression or activity level of the gene or the protein encoded thereby with the expression or activity level of a corresponding gene or a protein encoded thereby in a normal control sample; and
(c) co-administering a CXCL12 inhibitor, a CSF1 inhibitor, and a cancer immunotherapy agent when the expression or activity level of the gene or the protein encoded thereby is increased.

As used herein, the term “CXCL12 (C-X-C motif chemokine 12)”, also referred to as “stromal-cell-derived factor 1 (SDF1)”, is a ligand of the C-X-C chemokine receptor CXCR4 and is known to interact with CXCR4, and has also been reported as a ligand of CXCR7 (RDCI). CXCL12 may be broadly expressed in a variety of tissue types including the heart, liver, spleen, kidneys, brain, skeletal muscle, endothelial cells, epithelial tissue, stem cells, and the like. The activity of CXCL12 is associated with cellular functions including embryonic development, cell death and survival, immune response, tissue homeostasis, angiogenesis, calcium ion homeostasis, cell proliferation and migration, tumor growth and metastasis, and the like. CXCL12 is a strong chemoattractant for lymphocytes, and plays an important role in angiogenesis by recruiting endothelial progenitor cells from the bone marrow through a CXCR4-dependent mechanism. It is also known that metastasis of CXCR4+ tumor cells is involved in inducing metastasis to organs, such as lymph nodes, lungs, liver, and bones, which highly express CXCL12.

As used herein, the term “CSF1”, also known as macrophage colony-stimulating factor (M-CSF), is a secreted cytokine that differentiates hematopoietic stem cells into macrophages or other related cell types. Moreover, in general, eukaryotic cells produce and secrete M-CSF to fight intercellular virus infection, and the secreted MCSF binds to the CSF1 receptor and activates an intracellular signaling pathway. CSF1 is involved in proliferation, differentiation, and survival of monocytes, macrophages, and bone marrow progenitor cells.

In an embodiment of the present invention, it has been confirmed that the expression of the CXCL12 gene and the CSF1 gene is enhanced in cancer cells resistant to cancer immunotherapy agents, particularly p16^{INK4A}-overexpressing cancer cells. Furthermore, it has been confirmed that overexpression of the CXCL12 gene in cancer cells inhibits infiltration of CD8 cytotoxic T cells, and overexpression of the CSF1 gene is involved in the differentiation of M2-type macrophages to thus reduce the activity of cytotoxic T cells, thereby acquiring resistance to a cancer immunotherapy agent.

In the present invention, the CXCL12 inhibitor may be a CXCL12 gene expression inhibitor or a CXCL12 protein activity inhibitor.

In the present invention, the CSF1 inhibitor may be a CSF1 gene expression inhibitor or a CSF1 protein activity inhibitor.

As used herein, the term “gene expression inhibitor” refers to any agent that suppresses or inhibits expression of a target gene through transcription and translation. Any type of agent that specifically inhibits expression of a target gene may be included, and both inhibitors based on transcription regulation and post-transcriptional translation regulation, such as RNAi, are included. Examples of the gene expression inhibitor may include an antisense nucleotide, siRNA (small interfering RNA), shRNA (small hairpin RNA), ribozyme, aptamer, anti-microRNA, microRNA mimic, nucleases such as zinc finger nucleases (ZFNs) and transcription activator-like effector nucleases (TALENs), CRISPR-Cas9 systems (e.g. gRNA, sgRNA), polycistronic tRNA-gRNA systems, and self-ribozyme-flanked RNAs (Gao & Zhao 2014, Xie et al. 2015, Zetsche et al. 2017), but are not limited thereto. In the present invention, the target gene is a CXCL12 gene or a CSF1 gene.

The antisense nucleotide binds (hybridizes) to a complementary nucleotide sequence of DNA, immature mRNA, or mature mRNA to thus interfere with the flow of genetic information from DNA to protein. siRNA has an operating principle similar to the antisense nucleotide, but is double-stranded, 21-25 nucleotides long, and destroys mRNA through an enzyme complex called RISCs (RNA-induced silencing complexes), not RNase H. CRISPR-Cas9 (CRISPR gene scissors) acts as a restriction enzyme that recognizes a specific nucleotide sequence and cleaves DNA at the corresponding site.

As used herein, the term “protein activity inhibitor” refers to any agent that suppresses or inhibits the activity of a target protein. The protein activity inhibitor may directly inhibit the activity of the target protein or interfere with the interaction with other proteins to thereby inhibit the function thereof. The protein activity inhibitor includes, for example, a compound, a peptide, a peptidomimetic, a substrate analogue, an aptamer, and an antibody, each of which specifically binds to a protein, but is not limited thereto. In the present invention, the target protein is a CXCL12 protein or a CSF1 protein.

The compound includes any compound capable of specifically binding to a target protein and inhibiting the activity thereof. The aptamer is a single-stranded DNA or RNA molecule, and may be obtained by isolating an oligomer that binds with high affinity and selectivity to a specific chemical or biological molecule through an advanced method using an oligonucleotide library called SELEX (systematic evolution of ligands by exponential enrichment). The aptamer may specifically bind to a target protein and modulate the activity of the target protein by, for example, blocking the activity of the target protein through binding thereto. “Antibody” refers to a collection of antibody protein molecules including one or more complementarity-determining regions, an antibody protein molecule, a binding fragment, or a derivative thereof.

In the present invention, the term “inhibitor” may be used interchangeably with “antagonist” or “suppressor”.

As used herein, the term “target” used in the expression “target protein”, “target gene”, “targeting”, etc., refers to a material of interest, the activity of which is to be modulated. In particular, in the present invention, an inhibitor is characterized in that the expression or activity of a target gene of interest may be modulated.

In an embodiment of the present invention, an anti-CXCL12 antibody (Merck, Darmstadt, Germany, clone K15C) and an anti-CSF1 antibody (BioXcell, clone 5A1) were used as the CXCL12 inhibitor and the colony-stimulating factor 1 inhibitor, respectively.

In an embodiment of the present invention, the combined effect of the CXCL12 inhibitor and the CSF1 inhibitor with the immune checkpoint inhibitor (PD-1 inhibitor) was confirmed.

However, as confirmed in other embodiments of the present invention, considering that treatment resistance in p16^{INK4A}-overexpressing cancer is acquired through reduction of T-cell tumor infiltration and activity due to overexpression of CXCL12 and CSF1, it is possible to obtain a significant therapeutic enhancement effect as confirmed in Examples of the present invention when used in combination with a cancer immunotherapy agent or immunotherapy using T cells, in addition to the immune checkpoint inhibitor used.

Therefore, in the present invention, the cancer immunotherapy agent is capable of modulating the immune activity of T cells.

In the present invention, the cancer immunotherapy agent is preferably an immune checkpoint inhibitor or an immune cell therapy agent, more preferably an immune checkpoint inhibitor.

In the present invention, the immune checkpoint inhibitor may be characterized in that it targets at least one immune checkpoint selected from the group consisting of A2AR, B7-H3 (CD276) or B7-H3 receptor, B7-H4 (VTCN1) or B7-H4 receptor, BTLA (CD272), CTLA-4 (CD152), IDO, KIR, LAG3, NOX2, PD-1, PD-L1, PD-L2, TIM3, VISTA, and SIGLEC7, and is preferably a T-cell immune checkpoint inhibitor, more preferably a PD-1/PD-L1 pathway inhibitor such as a PD-1 inhibitor or a PD-L1 inhibitor, most preferably a PD-1 inhibitor.

In the present invention, the immune cell therapy agent may be selected from the group consisting of dendritic cell immunotherapeutic agents, LAK cell immunotherapeutic agents, T-cell-based immunotherapeutic agents, and NK-cell-based immunotherapeutic agents.

In the present invention, the immune cell therapy agent is preferably a T-cell-based immunotherapeutic agent. The T-cell-based immunotherapeutic agent may be a tumor-infiltrating T lymphocyte (TIL), a T-cell-receptor-modified T cell (TCR-T), or a chimeric-antigen-receptor-modified T cell (CAR-T), but is not limited thereto.

As used herein, the term “cancer immunotherapy agent” refers to a therapeutic agent that induces immune cells to selectively attack only cancer cells by stimulating the immune system through injection of an artificial immune protein into the body, unlike conventional anticancer agents that attack the cancer itself. In order to overcome the acquired immunosuppression or immune evasion mechanism in cancer cells, the cancer immunotherapy agent can be said to be a drug having a mechanism of restoring or strengthening the tumor recognition or destruction ability of the immune system. Cancer immunotherapy agents may be classified into those used for passive immunotherapy and those used for active immunotherapy. Passive immunotherapy includes an immune checkpoint inhibitor, an immune cell therapy agent, and a therapeutic antibody, and active immunotherapy includes a cancer treatment vaccine and an immune-modulating agent, but the present invention is not limited thereto. The cancer immunotherapy agent described above is well described in Issues & Trends on Cancer Immunotherapy Agents, published by the Korea Pharmaceutical Information Center.

As used herein, the term “immune checkpoint” refers to a molecule that regulates the activity of immune cells. The immune checkpoint exists for self-resistance, which prevents the immune system from attacking cells indiscriminately. The immune checkpoint may be classified into a stimulatory checkpoint that enhances immune activity and an inhibitory checkpoint that lowers immune activity. Some types of cancer protect themselves from immune responses by targeting inhibitory immune checkpoints.

As used herein, the term “immune checkpoint inhibitor” is a drug that blocks the activity of a target immune checkpoint protein involved in suppressing immune cells to thereby activate immune cells to attack cancer cells. Examples of the immune checkpoint that may be the target of the immune checkpoint inhibitor may include PD-1, PD-L1, CD80, CD86, CTLA4, B7-H3, -H4, -H5, BTLA, 4-1BB, Tim-3, TIGIT, CD94/NKG2A, KIR2DL-1, -2, -3, and the like, and targets of currently clinically useful immune checkpoint inhibitors are PD-1, PD-L1, and CTLA4. More specifically, currently clinically useful immune checkpoint inhibitors include CTLA-4 monoclonal antibodies such as ipilimumab and tremelimumab, PD-1 monoclonal antibodies such as nivolumab and pembrolizumab, PD-L1 monoclonal antibodies such as atezolizumab, durvalumab, and avelumab, and the like, but are not limited thereto.

As used herein, the term “immune cell therapy agent” refers to a cell therapy agent in which immune cells in the body are collected and strengthened or are genetically modified and placed back therein. Treatment using an immune cell therapy agent is called adoptive cell transfer (ACT). Immune cells used in the immune cell therapy agent include dendritic cells, lymphokine-activated killers (LAKs), and T cells (lymphocytes), depending on the characteristics of the genes introduced into the cells. Also, T cells are classified into tumor-infiltrating T lymphocytes (TILs), T-cell-receptor-modified T cells (TCR-T), chimeric-antigen-receptor-modified T cells (CAR-T), etc. Depending on the immune cells used in the immune cell therapy agent, the immune cell therapy agent may also be classified into a dendritic cell immunotherapeutic agent, a LAK cell immunotherapeutic agent, a T-cell-based immunotherapeutic agent, an NK-cell-based immunotherapeutic agent, or the like, but the present invention is not limited thereto.

Clinical drugs corresponding to immune cell therapy agents include sipuleucel-T (PROVENGE®), autologous dendritic cells (product name: CREAVAX-RCC®, JW CreaGene), activated T lymphocytes (product name: IMMUNOCELL-LC®, Green Cross), and tisagenlecleucel (product name: KYMRIAH®, Novartis), but are not limited thereto.

In an embodiment of the present invention, it has been confirmed that p16^{INK4A}-gene-overexpressing cancer cells overexpress CXCL12 and CSF1 and acquire resistance to cancer immunotherapy agents through respective T-cell action inhibition mechanisms, thus culminating in the present invention.

Therefore, in the present invention, the immune checkpoint inhibitor or immune cell therapy agent may be based on the activation of a T-cell-related immune response, preferably based on the activation of cytotoxic T cells or CD8 T cells.

The “CTLA-4” of the present invention is an antigen having a structure similar to that of CD28, and is a type of T-cell-activating antigen that is transiently expressed when T cells are activated. In order for T cells to be activated and display an immune response, antigen-presenting cells (APCs) must bind to T cells by sending two signals: MHC and B7.1/B7.2 (CD80/CD86). As such, when CTLA-4 binds to B7, the function of T cells is blocked, and the ability of T cells to recognize and kill cancer cells is blocked. Here, the CTLA-4 inhibitor binds to the CTLA-4 receptor, thus preventing inactivation of T cells and increasing the proliferation of T cells to activate T cells.

As used herein, the terms “PD-1” and “PD-L1” refer to proteins related to immune checkpoints that regulate the immune activity of CD8 T cells, and PD-L1 is a ligand of PD-1. PD-L1 is an immune evasion material mainly expressed on the surface of cancer cells. When PD-L1 binds to PD-1, T cells lose the function thereof and die. PD-1 inhibitors and PD-L1 inhibitors inhibit the expression of PD-1 and PD-L1, respectively, or inhibit the interaction to thus block the PD-1/PD-L1 immune evasion signaling pathway, thereby causing T cells to kill cancer cells.

In the present invention, each of the CXCL12 inhibitor, the CSF1 inhibitor, and the cancer immunotherapy agent may include one or more types thereof.

The “p16^{INK4A}” of the present invention is also called p16, cyclin-dependent kinase inhibitor 2A, CDKN2A, multiple tumor suppressor 1, etc., and is a protein that acts as a tumor suppressor by slowing cell division due to delayed progression of the cell cycle from G1 to S phase. p16^{INK4A}, which is a cancer inhibitory material, is known to accelerate the cell cycle and to cause many types of cancer when it is not sufficiently expressed or the function thereof is reduced or lost due to gene deletion or the like. Carcinomas known to have clinical significance in which p16^{INK4A} is overexpressed include breast cancer, gallbladder cancer, gastrointestinal stromal tumor, melanoma, and high-grade astrocytoma.

In an embodiment of the present invention, breaking away from the previously reported role of p16^{INK4A}, the infiltration of CD8 T cells into cancer tissues was reduced in proportion to the expression level of p16^{INK4A}, thereby confirming the correlation between expression of p16^{INK4A} and resistance to cancer immunotherapy agents. It was also confirmed that CXCL12 and CSF1 were overexpressed in the p16^{INK4A}-overexpressing cancer cells, which affects the infiltration of CD8 T cells in cancer cells and the differentiation of macrophages, thereby acquiring resistance to a cancer immunotherapy agent.

Accordingly, in the present invention, the cancer may be a cancer that is resistant to the cancer immunotherapy agent.

In the present invention, the cancer may be resistant to the immune checkpoint inhibitor.

In the present invention, the cancer may be characterized in that the expression of at least one gene selected from the group consisting of p16^{INK4A}, CXCL12, and CSF1 is increased compared to normal cells, and preferably, the expression of p16^{INK4A} is increased compared to normal cells.

For example, the cancer may be cancer with clinical significance in which p16^{INK4A} is overexpressed, and is preferably selected from the group consisting of colorectal cancer, breast cancer, gallbladder cancer, gastrointestinal stromal tumor, melanoma, and high-grade astrocytoma, and more preferably colorectal cancer.

In the present invention, the pharmaceutical composition may be a pharmaceutical composition for co-administration for the prevention and/or treatment of cancer including a CXCL12 inhibitor, a CSF1 inhibitor, and a cancer immunotherapy agent.

In the present invention, the pharmaceutical composition for co-administration may be in a form for simultaneous administration of three agents, including a mixture of a CXCL12 inhibitor, a CSF1 inhibitor, and a cancer immunotherapy agent.

In the present invention, the pharmaceutical composition may be in a form for simultaneous or sequential administration of three agents, in which each of a CXCL12 inhibitor, a CSF1 inhibitor, and a cancer immunotherapy agent is formulated. As such, the pharmaceutical composition for co-administration may be a pharmaceutical composition for co-administration including a first pharmaceutical composition including any one or two of a CXCL12 inhibitor, a CSF1 inhibitor, and a cancer immunotherapy agent as active ingredients, and a second pharmaceutical composition including the remainder, which are administered simultaneously or sequentially. For sequential administration, the sequence in which the agents are administered may be changed.

As used herein, the term “prevention” refers to any action of inhibiting or delaying the onset of cancer by administering the pharmaceutical composition of the present invention to a subject suspected of having developed cancer.

As used herein, the term “treatment” refers to any action of ameliorating or eliminating the symptoms of the disease by administering the pharmaceutical composition of the present invention to a subject suffering from cancer.

As used herein, the term “subject” refers to any animal, including a human, which has or is likely to develop cancer, preferably cancer that is resistant to a cancer immunotherapy agent. The animal may be a mammal, such as a cow, horse, sheep, pig, goat, camel, antelope, dog, cat, etc., in need of treatment for symptoms similar thereto, as well as humans, but is not limited thereto. In the present invention, the subject may be in need of prevention and/or treatment of cancer.

The pharmaceutical composition of the present invention includes a cancer immunotherapy agent, a CXCL12 inhibitor, and a CSF1 inhibitor, and exhibits significantly improved preventive and therapeutic effects on the above-described cancer, preferably cancer resistant to cancer immunotherapy agents, more preferably p16^{INK4A}-overexpressing cancer.

The pharmaceutical composition according to the present invention may contain, as an active ingredient, an antisense nucleotide, siRNA, shRNA, compound, natural material, extract, etc. capable of inhibiting the expression or activity of the gene or protein described above.

The pharmaceutical composition of the present invention may be used as a single formulation, or may be prepared and used as a combination formulation by additionally including a drug known to have a preventive and/or therapeutic effect on cancer, and may be used in combination with anticancer treatment other than the agent. In the present invention, examples of the cancer immunotherapy agent that may be used in combination therewith may include pembrolizumab, nivolumab, atezolizumab, avelumab, durvalumab, immune cell therapy agents based on T cells such as CAR T, and the like, but are not limited thereto.

The pharmaceutical composition of the present invention may be formulated into a unit dose form or placed in a multi-dose container using a pharmaceutically acceptable carrier or excipient.

As used herein, the term “pharmaceutically acceptable carrier” refers to a carrier or diluent that does not inhibit the biological activity or properties of the injected compound and does not irritate the organism. The type of carrier usable in the present invention is not particularly limited, and any carrier that is commonly used in the art and is pharmaceutically acceptable may be used. Non-limiting examples of the carrier include saline, sterile water, Ringer's solution, buffered saline, albumin injection solution, dextrose solution, maltodextrin solution, glycerol, ethanol, and the like, which may be used alone or in combination of two or more thereof. The carrier may include a non-naturally occurring carrier. In addition, if necessary, the pharmaceutical composition of the present invention may be used along with other typical additives such as antioxidants, buffers, and/or bacteriostats, and may be additionally added with diluents, dispersants, surfactants, binders, lubricants, etc. and may thus be used in the form of injectable formulations such as aqueous solutions, suspensions, emulsions, etc., pills, capsules, granules, or tablets.

The composition is usually formulated using a diluent or excipient such as a filler, an extender, a binder, a wetting agent, a disintegrant, a surfactant, and the like.

The pharmaceutical composition according to the present invention may be formulated and used in various forms according to typical methods. Suitable formulations include oral dosage forms such as tablets, pills, powders, granules, sugar-coated tablets, hard or soft capsules, solutions, suspensions, emulsions, injections, aerosols, etc., external preparations, suppositories, sterile injectable solutions, and the like, but the present invention is not limited thereto.

The pharmaceutical composition according to the present invention may be formulated in a suitable dosage form using a pharmaceutically inert organic or inorganic carrier. Specifically, when the formulation is a tablet, a coated tablet, a sugar-coated tablet, or a hard capsule, lactose, sucrose, starch or a derivative thereof, talc, calcium carbonate, gelatin, stearic acid or a salt thereof may be included. In addition, when the formulation is a soft capsule, it may contain vegetable oils, waxes, fats, semi-solids, and liquid polyols. In addition, when the formulation is in the form of a solution or syrup, water, polyol, glycerol, and vegetable oil may be included.

The pharmaceutical composition according to the present invention may further include a preservative, a stabilizer, a wetting agent, an emulsifier, a solubilizing agent, a sweetener, a colorant, an osmotic pressure regulator, an antioxidant, and the like, in addition to the carrier described above.

The pharmaceutical composition according to the present invention may be administered in a pharmaceutically effective amount. In the present invention, a “pharmaceutically effective amount” is an amount sufficient to treat a disease at a reasonable benefit/risk ratio applicable to medical treatment, and for the purposes of the present invention, the specific therapeutically effective amount for any particular patient typically varies depending on various factors including the type and extent of response to be achieved, the specific composition, including or not including other agents in some cases, the patient's age, body weight, general health, gender and diet, the time of administration, the route of administration, the rate of secretion of the composition, the treatment duration, and drugs used together with or concurrently with the specific composition, and similar factors well known in the medical art.

The pharmaceutical composition of the present invention may be administered as an individual therapeutic agent or in combination with other therapeutic agents, may be administered sequentially or simultaneously with conventional therapeutic agents, and may be administered in one or multiple doses. In consideration of all of the above factors, it is important to administer the composition in an amount and interval capable of obtaining the maximum effect using a minimum amount without side effects, which may be easily determined by those skilled in the art.

As used herein, the term “administration” refers to introduction of the pharmaceutical composition of the present invention to a patient by any suitable method, and the composition of the present invention may be administered through various routes, including oral or parenteral routes, so long as it is able to reach the target tissue. The mode of administration of the pharmaceutical composition according to the present invention is not particularly limited, and may follow a method commonly used in the art. The mode of administration may be performed in a typical manner through, for example, intravenous, intraarterial, intraperitoneal, intramuscular, intraarterial, intraperitoneal, intrasternal, transdermal, intranasal, pulmonary, topical, rectal, oral, intraocular, or intradermal routes, but the present invention is not limited to the above examples.

The pharmaceutical composition of the present invention is determined depending on the type of drug used as an active ingredient, as well as several related factors, such as the disease to be treated, the route of administration, the patient's age, gender, and body weight, and the severity of disease.

The frequency of administration of the composition of the present invention is not particularly limited, but the composition may be administered once a day or several times by dividing the dose.

In the present invention, the isolated biological sample may include tissues, cells, whole blood, serum, plasma, saliva, etc. that are different from the normal control in the expression or activity level of the gene or the protein, like the sample of the patient suspected of having disease, and is preferably colorectal cancer tissue, but is not limited thereto.

In the method of preventing and/or treating cancer according to the present invention, the CXCL12 inhibitor, the CSF1 inhibitor, and the cancer immunotherapy agent may be administered in combination in respective pharmaceutically effective amounts. The method may further include identifying a patient in need of prevention and/or treatment of cancer before administration.

In the present invention, the co-administration step may be performed by administering a mixture of a CXCL12 inhibitor, a CSF1 inhibitor, and a cancer immunotherapy agent. In the present invention, the co-administration step may include a first step of administering any one or two of a CXCL12 inhibitor, a CSF1 inhibitor, and a cancer immunotherapy agent as active ingredients and a second step of administering the remainder not administered in the first step, said steps being performed simultaneously or sequentially. For sequential administration, the sequence in which the agents are administered may be changed.

In the present invention, when the CXCL12 inhibitor, the CSF1 inhibitor, and the cancer immunotherapy agent are administered sequentially, the most suitable dose and administration interval may be easily determined by a person skilled in the art in consideration of the type and extent of response to be achieved, the specific composition, including or not including other agents in some cases, the patient's age, body weight, general health, gender, and diet, the time of administration, the route of administration, the rate of secretion of the composition, the treatment duration, and the like.

A better understanding of the present invention may be obtained through the following examples. These examples are merely set forth to illustrate the present invention, and are not to be construed as limiting the scope of the present invention, as will be apparent to those skilled in the art.

### Examples

A better understanding of the present invention may be obtained through the following examples. These examples are merely set forth to illustrate the present invention, and are not to be construed as limiting the scope of the present invention, as will be apparent to those skilled in the art.

Colorectal Cancer Sample

Colorectal cancer (CRC) samples were obtained at Ajou University Hospital through surgical resection from patients who provided informed consent. Fresh tumorous tissue and normal tissue for SA-β-Gal staining were sampled separately from the representative area by an experienced pathologist immediately after dissection and partitioned into two identical tissue fragments. According to the tissue processing regulations of Ajou University Hospital, one was immediately frozen in liquid nitrogen, and the remaining one was treated with FFPE. Patients who had chemotherapy or radiation therapy before surgery were excluded from the study.

Cancer Cell Line

SW480 cells were purchased from the Korean Cell Line Bank (KCLB, Seoul, Korea). SW480 cells were maintained in a complete RPMI medium containing 10% FBS, and MC38 cells were purchased from Kerafast (Massachusetts, USA) and maintained in a complete DMEM containing 10% FBS.

Animal (Mouse) Experiment

MC38-control cells (1×10⁶), MC38-mouse CXCL12 cells (MC38-mCXCL12, 1×10⁶), MC38-mouse CSF1 cells (MC38-mCSF1, 1×10⁶), MC38-mouse shCSF1 cells (MC38-shmCSF1, 1×10⁶), CT26-control Cells (1×10⁶), or CT26-mouse CXCL12 cells (1×10⁶) were resuspended in 100 μL of PBS and subcutaneously injected into female or male, C57BL/6 or BALB/c mice (7 weeks of age), and the mice were sacrificed after 3 weeks. For the immune checkpoint inhibitor treatment trials, one week after injection of tumor cells (2×10⁶), a normal isotype immunoglobulin G (mouse IgG2a; clone C1.18.4, rat IgG2a; clone 2A3, rat IgG1k; clone HRPN, BioXcell, West Lebanon, NH) or anti-PD-1 antibody (10 mg/kg, BioXcell, clone RMP1-14) was intraperitoneally injected twice a week for 2 weeks. Mice having intraperitoneal tumors (4 mice; MC38/IgG, 1 mouse; MC38/PD1 Ab, 3 mice; MC38-mCXCL12/IgG, MC38-mCXCL12/PD1 Ab) were excluded. For the CXCL12 and CSF1 inhibitor treatment trials, the animal model system (Sanchez-Paulete et al., 2016; Woo et al., 2012) was partially modified. MC38-mCXCL12 cells (2×10⁶) were resuspended in 100 μL of PBS and injected subcutaneously into female C57BL/6 mice (7 weeks of age). After one week, a normal isotype immunoglobulin G (control-IgG) or anti-PD-1 antibody was intraperitoneally injected alone or in combination with an anti-CXCL12 (500 μg/kg, Merck, Darmstadt, Germany, clone K15C) or anti-CSF1 (15 mg/kg, BioXcell, clone 5A1) neutralizing antibody twice a week for 2 weeks.

Tumor volume was measured with a caliper and calculated using the following equation:

V=(width²×length)/2

Immunohistochemistry staining and immunocytochemistry staining

Immunohistochemistry staining was performed on 4-μm-thick representative tissue fragments of formalin-fixed paraffin-embedded tissue using a Benchmark XT automated processor (Ventana Medical Systems Inc., Tucson, AZ). Detection was performed using a Ventana Optiview DAB kit (Ventana Medical Systems). Dual immunohistochemistry assay was performed using an UltraView universal DAB detection kit (#760-500, Ventana Medical Systems Inc) for the first antibody in a Benchmark XT automated immunohistochemistry stainer and an UltraView Universal Alkaline Phosphatase Red detection kit (#760-501, Ventana Medical Systems Inc) for the second antibody.

Immunocytochemistry staining was performed by washing the slides twice with PBS and culturing the same for 1 hour with an appropriately conjugated secondary antibody. For F-actin staining, rhodamine phalloidin was applied to the slides for 1 hour, followed by observation using a fluorescence microscope. When the cytoplasm or nucleus showed moderate or strong staining intensity, a positive result was recorded, and when there was little or no cytoplasm or nuclear staining, a negative result was recorded. Immunostaining for p16^{INK4A}, CXCL12, and CSF1 was graded depending on the proportion of immunopositive cells (0: less than 1%; 1+: 1-20%; 2+: 20-40%; and 3+: greater than 40%).

Cell Migration Analysis

Cell migration was assessed with a TRANSWELL® (5 μm pore size, 24 well, Corning, NY). The migrated cells were counted as suspension cells in the lower chamber.

ELISA Analysis

Cells (3×10⁵) were seeded in a 24-well plate and cultured for 48 hours, after which the medium was harvested. Secretion of CXCL12 or CSF1 into the culture medium was measured using a CXCL12 (DSA00, R&D Systems) or CSF1 ELISA kit (RayBiotech Life, Peachtree Corners, GA) according to the manufacturer's instructions.

FACS Analysis

Monocytes were cultured with mouse anti-human CD68-FITC (562117, 1:40, BD Biosciences, San Jose, CA) and mouse anti-human CD206-APC (550889, 1:40, BD Biosciences). After culture for 30 minutes in a dark room at room temperature, the cells were transferred to a 5 ml polystyrene round-bottom tube, and flow cytometry (BD FACSCanto II; BD Biosciences) was performed.

Monocyte Differentiation

Isolated primary monocytes were co-cultured with control, ROS-treated, CXCL12-overexpressing or CSF1-overexpressing SW480 cells using a TRANSWELL® (0.4 μm pore size, 6 wells, Corning), or co-cultured with a control (SW480)-derived conditioned medium or senescent tumor cells (ROS-induced senescent SW480). After culture for 6 days, the upper chamber was removed, the monocytes were isolated as single cells, and FACS and real-time PCR analysis were performed.

### Example 1: Confirmation of p16^{INK4A} Expression in Colorectal Cancer

The expression of p16^{INK4A} was confirmed through immunohistochemistry staining using colorectal cancer samples from colorectal cancer patients.

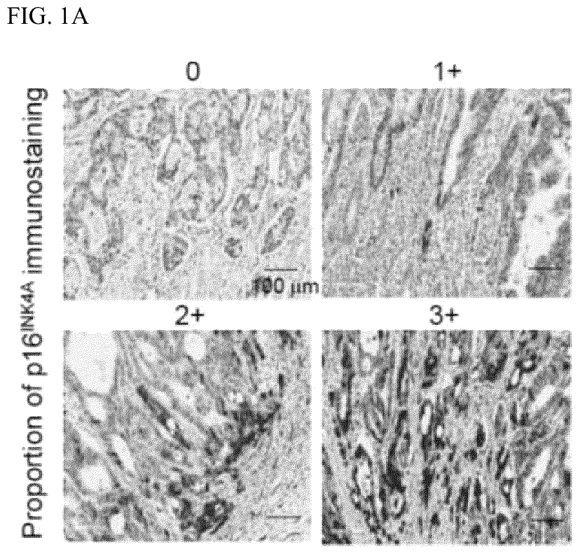

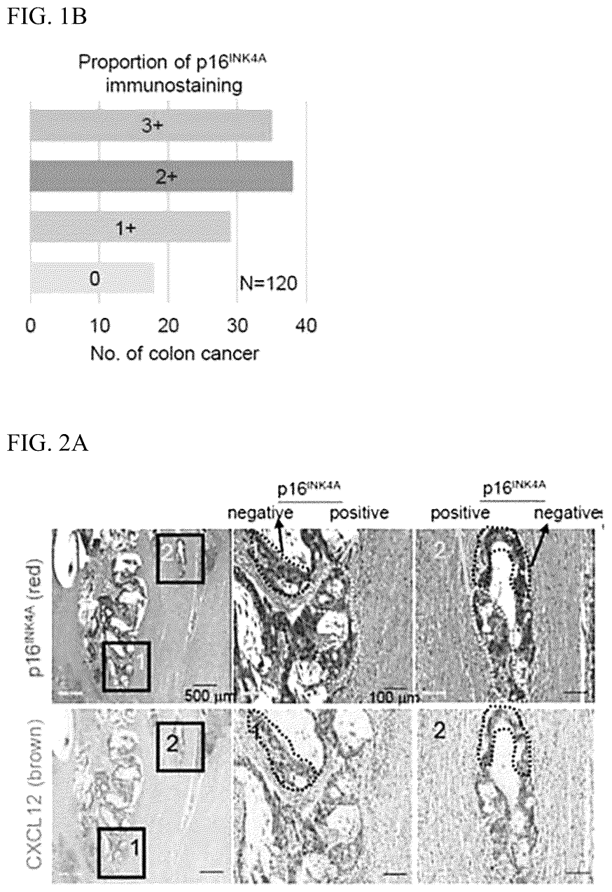

Immunochemistry staining was performed using 4-μm-thick paraffin-treated tissue fragments. Based on the results of immunohistochemistry staining of p16^{INK4A} analyzed through immunohistochemistry staining of 120 colorectal cancer samples, classification was performed depending on the proportion of stained cells (0: less than 1%, 1+: 1-20%, 2+: 20-40%, and 3+: greater than 40% (FIG. 1A)).

As shown in FIG. 1, it was confirmed that about 85% or more of colorectal cancer exhibited high p16^{INK4A} expression.

### Example 2: Confirmation of Expression of CXCL12 and CSF1 in p16^{INK4A}-Overexpressing Colorectal Cancer

Next, the expression of CXCL12 and CSF1 in p16^{INK4A}-overexpressing colorectal cancer was confirmed through immunochemistry staining. As in Example 1, immunochemistry staining was performed using 4-μm-thick paraffin-treated tissue fragments, and the antibody of Table 1 was used as a primary antibody.

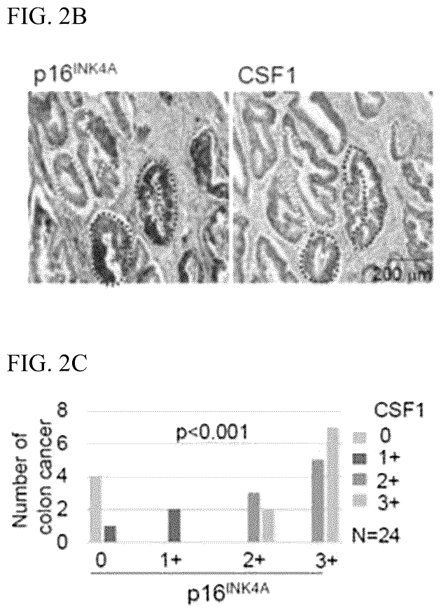

As shown in FIG. 2A, it can be confirmed that the negative (black dotted line) or positive (yellow dotted line) p16^{INK4A} expression regions of colorectal cancer tissue matched the negative (black dotted line) or positive (yellow dotted line) CXCL12 expression regions, which means that the expression of CXCL12 was increased in p16^{INK4A}-expressing colorectal cancer cells.

As shown in FIG. 2B, it can be confirmed that the negative (red dotted line) or positive (yellow dotted line) p16^{INK4A} expression regions of colorectal cancer tissue matched the negative (red dotted line) or positive (yellow dotted line) CSF1 expression regions, which means that the expression of CSF1 was increased in p16^{INK4A}-expressing colorectal cancer cells.

As shown in FIG. 2C, it was confirmed that the expression level of CSF1 was also increased with the increase in the expression level of p16^{INK4A} in the colorectal cancer samples (horizontal axis).

### Example 3: Confirmation of Mechanism of Acquired Resistance to Cancer Immunotherapy Agent in p16^{INK4A}-Overexpressing Colorectal Cancer

### Example 3-1: Immune Evasion of Colorectal Cancer by CXCL12

Control tumor cells (MC38-control) and CXCL12-overexpressing tumor cells (MC38-mCXCL12) were subcutaneously injected into 10 mice (7 weeks of age) in the same manner as described in the animal (mouse) experiment, and changes in tumor size were measured. Then, infiltration of CD8 T cells in the isolated tumors was analyzed.

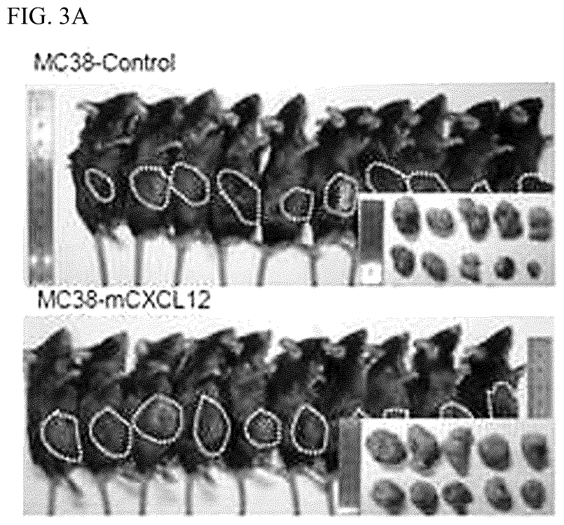

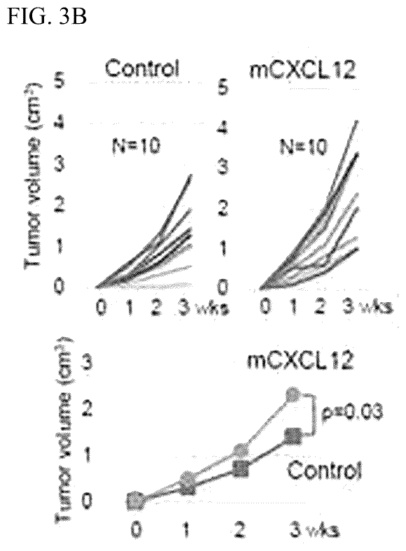

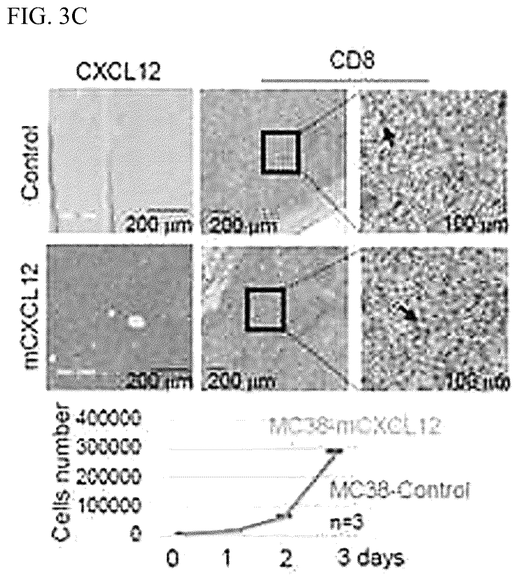

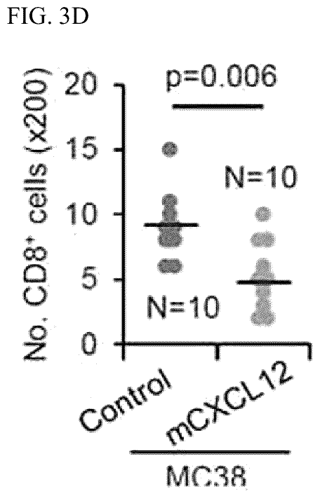

As shown in FIGS. 3A and 3B, in the mice injected with CXCL12-overexpressing tumor cells, the rate of increase in tumor size was much greater.

FIG. 3C shows the rates of growth of two cell groups when the control and CXCL12-overexpressing cell lines were cultured in vitro in cell culture dishes. The rates of growth of both cell lines were the same, which means that the difference in the size of tumors occurring after subcutaneous injection of the cell line into the mice is not due to the self-division rate of each cell line, but is due to the extent of infiltration of CD8 T cells in the environment surrounding the tumor.

As shown in FIG. 3D, it can be confirmed that infiltration of T cells in the CXCL12-secreting tumors was significantly reduced.

Furthermore, through immunochemistry staining analysis, when a CXCL12 antibody, a CSF1 antibody, and a PD-1 antibody were administered alone or in combination, intratumoral infiltration of CD8 T cells was confirmed in CXCL12-overexpressing tumor cells (MC38-mCXCL12).

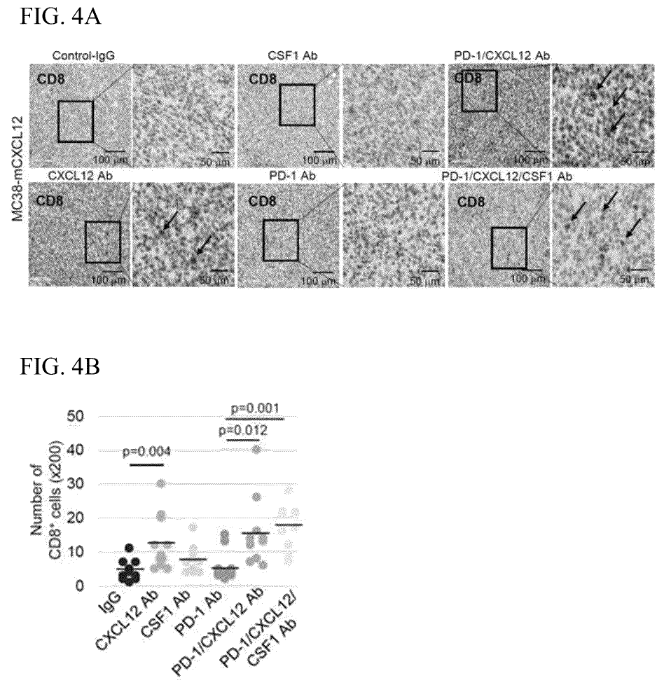

As shown in FIG. 4, single administration of the CSF1 antibody or the PD-1 antibody did not cause a significant increase in CD8 T-cell infiltration in the tumors compared to the control (IgG), and CD8 T-cell infiltration was significantly increased upon administration of the CXCL12 antibody alone, co-administration of CXCL12/PD-1 antibody, and co-administration of CXCL12/CSF1/PD-1 antibody. In particular, the greatest CD8 T-cell infiltration appeared upon co-administration of CXCL12/CSF1/PD-1.

### Example 3-2: Immune Evasion of Colorectal Cancer by CSF1

In order to confirm the mechanism of acquired resistance to a cancer immunotherapy agent in colorectal cancer by CSF1, the isolated primary monocytes were co-cultured with control, ROS-treated, CXCL12-overexpressing, CSF1-overexpressing, or CXCL12/CSF1-overexpressing SW480 cells using a TRANSWELL® (0.4 μm pore size, 6 wells, Corning), or co-cultured with a control (SW480)-derived conditioned medium or senescent tumor cells (ROS-induced senescent SW480). After culture for 6 days, the upper chamber was removed, the monocytes were isolated as single cells, and FACS and real-time PCR analysis were performed.

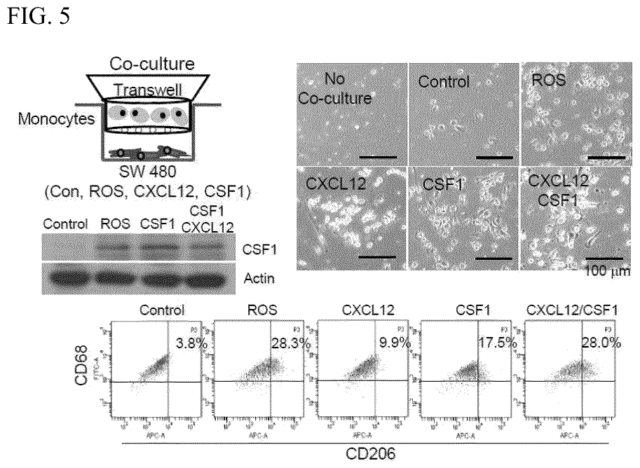

As shown in FIG. 5, it was confirmed that SW480 cells overexpressing CXCL12, overexpressing CSF1, or overexpressing both CXCL12 and CSF1 promoted the differentiation of monocytes into M2-type macrophages (CD68+CD206+) compared to the control. In particular, the SW480 cells overexpressing both CXCL12 and CSF1 exhibited the same level of ability to induce macrophage differentiation as those directly treated with ROS.

M2-type macrophages (CD68+CD206+) are tumor-promoting macrophages and are known to promote tumor growth by reducing the activity of cytotoxic T cells. Additionally, control tumor cells (MC38), CSF1-overexpressing tumor cells (MC38-CSF1), and CSF1-inhibiting tumor cells (MC38-CSF1) were subcutaneously injected into mice, and tumor growth was observed.

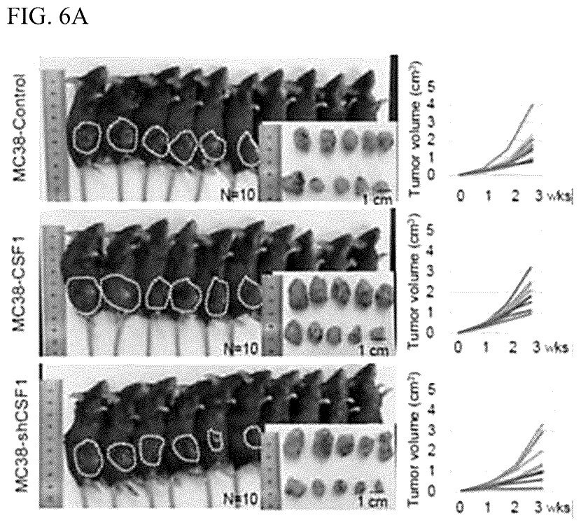

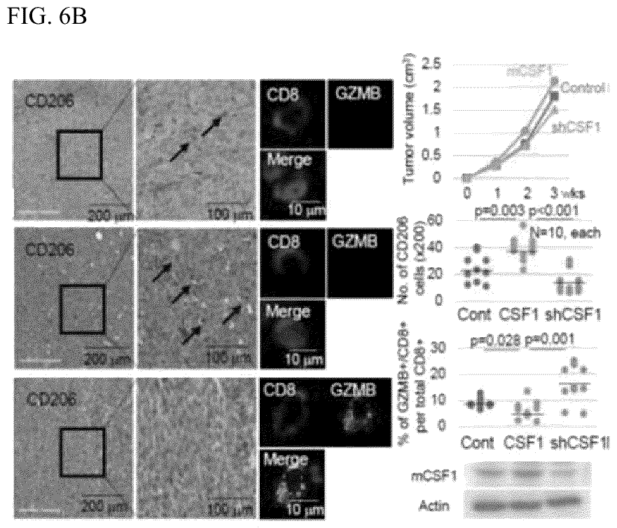

As shown in FIG. 6A, in the CSF1-overexpressing tumor cells (MC38-CSF1), tumor growth was increased compared to the control, whereas in the CSF1-inhibiting tumor cells (MC38-shCSF1), tumor growth was decreased compared to the control.

Additionally, as shown in FIG. 6B, it was confirmed that the number of M2-type macrophages (CD206+) was increased and the number of granzyme B (GZMB) positive T cells, indicating the activity of CD8 T cells, was decreased through immunochemistry staining analysis. On the other hand, for the CSF1-inhibiting tumor cells (MC38-shCSF1), it was confirmed that the number of M2-type macrophages (CD206+) was decreased and the number of granzyme B (GZMB) positive T cells, indicating the activity of CD8 T cells, was increased.

Based on the results of Examples 3-1 and 3-2, the expression of CXCL12 and CSF1 was significantly increased in p16^{INK4A}-Overexpressing colorectal cancer, and the increased CXCL12 expression inhibited the infiltration of CD8 cytotoxic T cells in the tumors, and increased CSF1 was involved in the differentiation of monocytes into M2-type macrophages, thereby reducing the activity of cytotoxic T cells, confirming that resistance to T-cell-related immunity was acquired due to the synergistic effect of the two mechanisms.

The above result proves the mechanism of acquired resistance to a cancer immunotherapy agent related to the enhancement of T-cell efficacy in p16^{INK4A}-overexpressing cancer.

### Example 4: Confirmation of Effect of Co-Administration of CXCL12 Inhibitor, CSF1 Inhibitor, and Cancer Immunotherapy Agent on Cell Line Resistant to Cancer Immunotherapy Agent

Based on the mechanism of acquired resistance to a cancer immunotherapy agent related to the enhancement of T-cell efficacy in p16^{INK4A}-Overexpressing colorectal cancer identified in Example 3, the effect of co-administration of a CXCL12 inhibitor, a CSF1 inhibitor, and a cancer immunotherapy agent was confirmed through animal experiments.

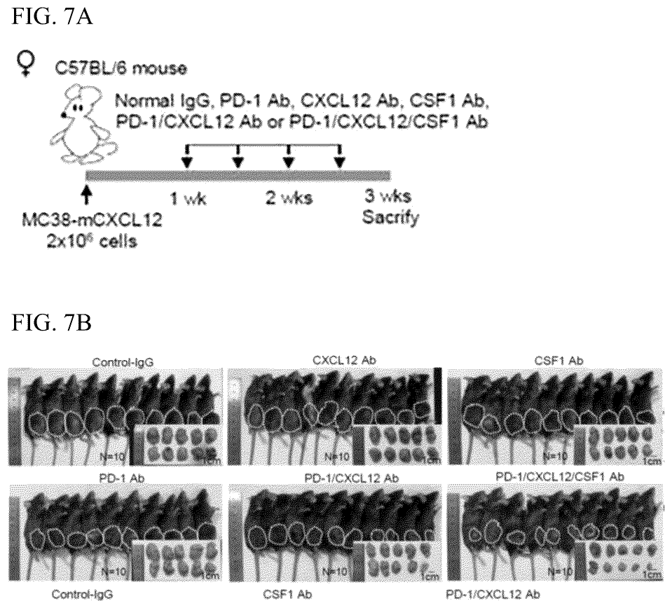

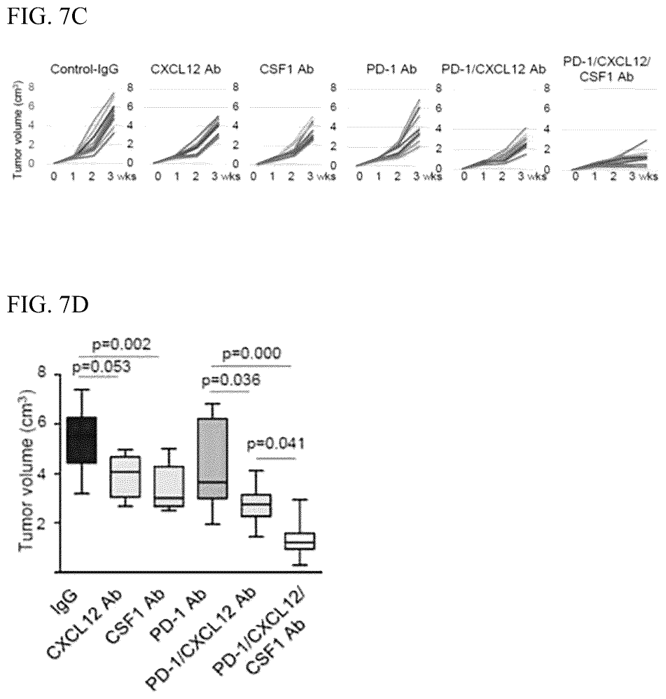

Female C57BL/6 mice were injected subcutaneously (7 weeks of age). After one week, a normal isotype immunoglobulin G (control-IgG) or anti-PD-1 antibody was intraperitoneally injected alone or in combination with an anti-CXCL12 (500 μg/kg, Merck, Darmstadt, Germany, clone K15C) or anti-CSF1 (15 mg/kg, BioXcell, clone 5A1) neutralizing antibody twice a week for 2 weeks, and in the 3^{rd} week, tumor cells were isolated to confirm the tumor size reduction effect (FIG. 7A).

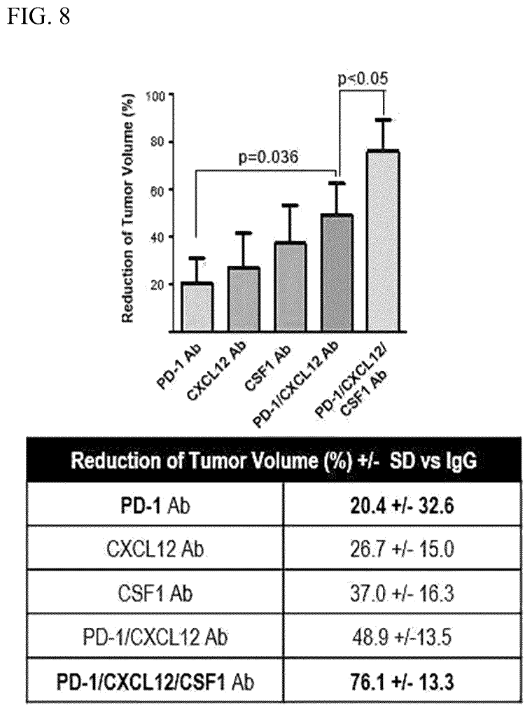

As shown in FIGS. 7 and 8, compared to the control, when the PD-1 antibody, the CXCL12 antibody, and the CSF1 antibody were administered alone, the tumor sizes were decreased by averages of 20.4%, 26.7%, and 37.0%, respectively, and co-administration of the PD-1 antibody and the CXCL12 antibody showed a tumor size reduction rate that was slightly increased to 48.9%. Co-administration of the PD-1 antibody, the CXCL12 antibody, and the CSF1 antibody showed an average tumor size reduction rate of 76.1%, indicating that the tumor size reduction rate was increased 2 to 3 times compared to single administration and 1.5 times or more compared to co-administration of CXCL12 and PD-1 antibodies.

As confirmed in this Example, co-administration of the PD-1 antibody, the CXCL12 antibody, and the CSF1 antibody exhibited significantly improved tumor size reduction and therapeutic effect against cancer that is resistant to a cancer immunotherapy agent. The pharmaceutical composition, combination therapy, and cancer treatment method according to the present invention have excellent effects in the treatment of cancer, particularly cancer that is resistant to cancer immunotherapy agents such as immune checkpoint inhibitors.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention is to be defined by the appended claims and equivalents thereof.

### INDUSTRIAL APPLICABILITY

According to the present invention, SP-HAMP technology according to the present invention is simple because it does not require separate primers of a complex design necessary for existing LAMP technology, has improved detection efficiency compared to conventional LAMP responses, and detects not only DNA but also RNA as a target nucleic acid, and is thus more widely applicable.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention is to be defined by the appended claims and equivalents thereof.

1-19. (canceled)

20. A method of preventing and/or treating cancer in a subject in need thereof, comprising co-administering a CXCL12 (C-X-C motif chemokine 12) inhibitor, a CSF1 (colony-stimulating factor 1) inhibitor, and a cancer immunotherapy agent to the subject.

21. The method according to claim 20, wherein the CXCL12 inhibitor is a CXCL12 gene expression inhibitor or a CXCL12 protein activity inhibitor.

22. The method according to claim 21, wherein the CXCL12 gene expression inhibitor is selected from the group consisting of an antisense nucleotide, siRNA (small interfering RNA), shRNA (small hairpin RNA), ribozyme, aptamer, anti-microRNA, microRNA mimic, nuclease, zinc finger nuclease (ZFN), transcription activator-like effector nuclease (TALEN), gRNA, sgRNA, and self-ribozyme-flanked RNA, each of which targets a CXCL12 gene.

23. The method according to claim 21, wherein the CXCL12 protein activity inhibitor is selected from the group consisting of a compound, a peptide, a peptidomimetic, a substrate analogue, an aptamer, an antibody, and a fragment thereof, each of which specifically binds to a CXCL12 protein.

24. The method according to claim 20, wherein the CSF1 inhibitor is a CSF1 gene expression inhibitor or a CSF1 protein activity inhibitor.

25. The method according to claim 24, wherein the CSF1 gene expression inhibitor is selected from the group consisting of an antisense nucleotide, siRNA (small interfering RNA), shRNA (small hairpin RNA), ribozyme, aptamer, anti-microRNA, microRNA mimic, nuclease, zinc finger nuclease (ZFN), transcription activator-like effector nuclease (TALEN), gRNA, sgRNA, and self-ribozyme-flanked RNA, each of which targets a CSF1 gene.

26. The method according to claim 24, wherein the CSF1 protein activity inhibitor is selected from the group consisting of a compound, a peptide, a peptidomimetic, a substrate analogue, an aptamer, an antibody, and a fragment thereof, each of which specifically binds to a CSF1 protein.

27. The method according to claim 20, wherein the cancer immunotherapy agent is an immune checkpoint inhibitor or an immune cell therapy agent.

28. The method according to claim 27, wherein the immune checkpoint inhibitor is a T-cell immune checkpoint inhibitor.

29. The method according to claim 27, wherein the immune checkpoint inhibitor targets an immune checkpoint selected from the group consisting of A2AR, B7-H3 (CD276), B7-H3 receptor, B7-H4 (VTCN1), B7-H4 receptor, BTLA (CD272), CTLA-4 (CD152), IDO, KIR, LAG3, NOX2, PD-1, PD-L1, PD-L2, TIM3, VISTA, SIGLEC7, and a combination thereof.

30. The method according to claim 27, wherein the immune checkpoint inhibitor targets PD-1 or PD-L1.

31. The method according to claim 27, wherein the immune cell therapy agent is a T-cell-based immunotherapeutic agent.

32. The method according to claim 20, wherein the cancer is a cancer that is resistant to the cancer immunotherapy agent.

33. The method according to claim 32, wherein an expression or activity level of a gene selected from the group consisting of p16^{INK4A}, CXCL12, CSF1, and a combination thereof, or a protein encoded thereby is increased in the cancer.

34. The method according to claim 33, wherein the expression of p16^{INK4A} is increased in the cancer compared to normal cells.

35. A method of preventing and/or treating cancer, comprising:
(a) measuring an expression or activity level of a gene selected from the group consisting of p16^{INK4A}, CXCL12, CSF1, and a combination thereof, or a protein encoded thereby in a biological sample isolated from a test subject;
(b) predicting cancer immunotherapy agent resistance of the test subject by comparing the expression or activity level of the gene or the protein encoded thereby with an expression or activity level of a corresponding gene or a protein encoded thereby in a normal control sample; and
(c) co-administering to the test subject a CXCL12 inhibitor, a CSF1 inhibitor, and a cancer immunotherapy agent when the expression or activity level of the gene or the protein encoded thereby in the biological sample of the test subject is higher than that of the normal control sample.
